(19) United States
(12) Patent Application Publication
(10) Pub. No.: US 2023/0307113 A1
(43) Pub. Date: Sep. 28, 2023

(54) RADIATION TREATMENT PLANNING USING MACHINE LEARNING

(71) Applicant: Siemens Healthineers International AG, Steinhausen (CH)

(72) Inventors: Esa Kuusela, Espoo (FI); Elena Czeizler, Helsinki (FI)

(21) Appl. No.: 17/704,507

(22) Filed: Mar. 25, 2022

(51) Int. Cl. G16H 20/40; G16H 50/70

(52) U.S. Cl. CPC G16H 20/40; G16H 50/70

(57) ABSTRACT

A control circuit accesses a plurality of previously-optimized radiation treatment plans and also accesses a plurality of optimization precursor information items. Each of the latter corresponds to at least one of the plurality of previously-optimized radiation treatment plans. The control circuit then generates a machine learning model using the plurality of previously-optimized radiation treatment plans and the plurality of optimization precursor information items. By one approach, at least a majority of the plurality of optimization precursor information items originate with a given radiation treatment facility and not with an unrelated (physically or institutionally) facility. These teachings will accommodate use of any of a variety of optimization precursor information items. By one approach, at least some of the plurality of optimization precursor information items comprise clinical goals.

### TECHNICAL FIELD

These teachings relate generally to treating a patient's planning target volume with energy pursuant to an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often generated through a so-called optimization process. As used herein, “optimization” will be understood to refer to improving a candidate treatment plan without necessarily ensuring that the optimized result is, in fact, the singular best solution. Such optimization often includes automatically adjusting one or more treatment parameters (often while observing one or more corresponding limits in these regards) and mathematically calculating a likely corresponding treatment result to identify a given set of treatment parameters that represent a good compromise between the desired therapeutic result and avoidance of undesired collateral effects.

Typical radiation treatment plan creation relies heavily upon an expert to manually guide the planning process (even when utilizing optimization algorithms). The applicant has determined that such a planner will likely focus more on some features than others. For example, while exposing normal tissue to radiation is considered potentially harmful, some organs exhibit greater sensitivity to radiation in a non-linear manner.

One approach to assist in the planning process uses a dose prediction model to facilitate optimization. That dose prediction model may comprise a machine learning model that is trained with historically accepted, high-quality plans. Unfortunately, though perhaps adequate for some purposes, such an approach does not always meet the needs of all application setting requirements.

### BRIEF DESCRIPTION OF THE DRAWINGS

The above needs are at least partially met through provision of the method and apparatus pertaining to radiation treatment planning using machine learning described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a graph as configured in accordance with various embodiments of these teachings; and

FIG. 4 comprises a schematic representation as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments a control circuit accesses a plurality of previously-optimized radiation treatment plans and also accesses a plurality of optimization precursor information items. Each of the latter corresponds to at least one of the plurality of previously-optimized radiation treatment plans. The control circuit then generates a machine learning model using the plurality of previously-optimized radiation treatment plans and the plurality of optimization precursor information items.

By one approach, at least a majority, or even substantially all (or all) of the plurality of optimization precursor information items originate with a given radiation treatment facility and not with an unrelated (physically or institutionally) facility.

These teachings are flexible in practice and will accommodate use of any of a variety of optimization precursor information items. By one approach, at least some of the plurality of optimization precursor information items comprise clinical goals. By another approach, in lieu of the foregoing or in combination there with, at least some of the plurality of optimization precursor information items comprise optimization objectives.

By one approach, generating the machine learning model comprises, at least in part, evaluating dose distributions in the aforementioned plurality of previously-optimized radiation treatment plans as a function of the plurality of optimization precursor information items to thereby identify emphasized features. Examples of emphasized features include, but are not limited to, a feature corresponding to an organ-at-risk protection compromise, a feature corresponding to a compromise between target coverage and organ-at-risk protection, and a feature corresponding to at least one spatially restricted area that has particular weight in achieving or failing a precursor specification.

By one approach, these teachings will accommodate generating the machine learning model using the plurality of previously-optimized radiation treatment plans and the plurality of optimization precursor information items as a training corpus by, at least in part, selecting a loss function to be minimized during training of the machine learning model.

These teachings are practical in application and will further accommodate, for example, optimizing a new radiation treatment plan as a function, at least in part, of the machine learning model.

So configured, these teachings provide for determining which features were likely to have been emphasized (or not) when generating previous radiation treatment plans and to leverage that information when training a machine learning model. Such an approach leverages previously exhibited emphasis by clinicians who were presumably adhering to clinical protocols for a given radiation treatment facility. That adherence, in turn, presumably reflected local quality-based goals.

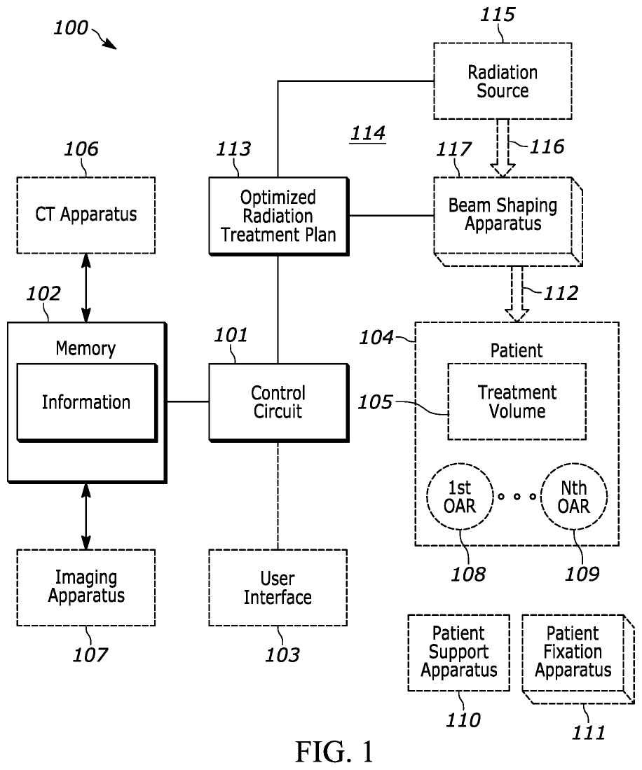

These and other benefits may become clearer upon making a thorough review and study of the following detailed description. Referring now to the drawings, and in particular to FIG. 1, a given radiation treatment facility that includes an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as radiation dosing information, previously-optimized radiation treatment plans, and a plurality of optimization precursor information items, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized radiation treatment plan 113. This radiation treatment plan 113 typically comprises specified values for each of a variety of treatment-platform parameters during each of a plurality of sequential exposure fields. In this case the energy-based treatment plan 113 is generated through an optimization process. Various automated optimization processes specifically configured to generate such an energy-based treatment plan are known in the art. As the present teachings are not overly sensitive to any particular selections in these regards, further elaboration in these regards is not provided here except where particularly relevant to the details of this description.

By one approach the control circuit 101 can operably couple to an energy-based treatment platform 114 that is configured to deliver therapeutic energy 112 to a corresponding patient 104 in accordance with the optimized energy-based treatment plan 113. These teachings are generally applicable for use with any of a wide variety of energy-based treatment platforms/apparatuses.

In a typical application setting the energy-based treatment platform 114 will include an energy source 115 such as a source of ionizing radiation, a source of microwave energy, a source of heat energy, and so forth. For the sake of an illustrative example, this description will presume the energy source 115 to comprise a source of high-energy radiation such as X-rays.

By one approach this energy source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the energy source 115 along that arcuate pathway, and may accordingly control when the energy source 115 starts moving, stops moving, accelerates, de-accelerates, and/or a velocity at which the energy source 115 travels along the arcuate pathway.

A typical radiation treatment platform 114 may also include one or more support apparatuses 110 (such as a couch) to support the patient 104 during the treatment session, one or more patient fixation apparatuses 111, a gantry or other movable mechanism to permit selective movement of the energy source 115, and one or more energy-shaping apparatuses 117 (for example, beam-shaping apparatuses such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

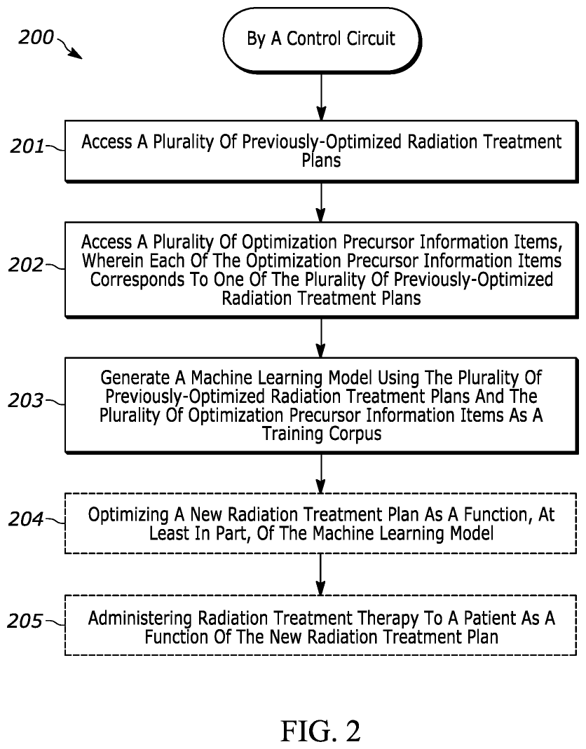

Referring now to FIG. 2, a process 200 that can be carried out, for example, in conjunction with the above-described application setting (and more particularly via the aforementioned control circuit 101) will be described.

At block 201, the control circuit 101 accesses a plurality of previously-optimized radiation treatment plans. By one approach some or all of these radiation treatment plans were actually used to administer therapeutic radiation. If desired, some or all of these radiation treatment plans, although optimized, were not actually used to administer therapeutic radiation. By one approach, at least most, or even all of these previously-optimized radiation treatment plans were optimized and/or utilized at a single given treatment facility (or at a plurality of treatment facilities that are institutionally related, for example, by a common owner). By another approach, at least some of the previously-optimized radiation treatment plans originated from one or more non-related sources such as other unrelated treatment facilities, medical research facilities (including academic institutions), manufacturers and/or suppliers of radiation treatment equipment, or otherwise.

At block 202, the control circuit 101 accesses a plurality of optimization precursor information items. Each of these optimization precursor information items corresponds to at least one of the plurality of previously-optimized radiation treatment plans. By one approach at least some of the optimization precursor information items each correspond to only one of the previously-optimized radiation treatment plans. By another approach, at least one of the optimization precursor information items corresponds to at least two of the plurality of previously-optimized radiation treatment plans.

By one approach, at least the majority of the plurality of optimization precursor information items originated with a given radiation treatment facility (or even only a single prescribing medical service provider). If desired, at least substantially all (or even all) of the plurality of optimization precursor information items originated with the given radiation treatment facility. That said, just as some of the previously-optimized radiation treatment plans may originate elsewhere, corresponding optimization precursor information items may similarly originate elsewhere.

These teachings will accommodate a variety of optimization precursor information items. Generally speaking, these items are information items that serve as direct and/or indirect inputs to the optimization process. In that way, these items are precursors to the optimization process itself. Examples include, but are not limited to, clinical goals and optimization objectives.

Clinical goals are the treatment goals being prescribed by, for example, an attending oncologist. Examples of clinical goals include, but are not limited to, goals regarding the dose distributions to be achieved with respect to a target volume, one or more organs-at-risk (OAR) in the vicinity of the target volume, or other specified or unspecified normal tissues. By their very nature, clinical goals are typically agnostic with respect to what physical radiation treatment platform serves to administer the radiation.

Optimization objectives provide a measure by which the process can test or assure that a particular specified dose is being uniformly administered through the patient's target volume while avoiding undue dosing of other patient tissues (or, in other cases, that a series of dose histograms that specify acceptable dosing ranges for a variety of locations both in and external to the target volume are met).

Accordingly, optimization objectives will be understood to be objectives that are very much specifically designed to reflect and accommodate the technical details and specifications of a particular radiation treatment platform, specific details regarding the patient's presentation, and/or other physical details pertaining to a particular application setting. Such details are generally viewed as being outside the expertise and knowledge base of the person who prescribes the radiation treatment in the first place (i.e., for example, a licensed oncologist). As a result, the person prescribing the radiation treatment ordinarily does not also create the optimization objectives.

At block 203, the control circuit 101 generates a machine learning model using the plurality of previously-optimized radiation treatment plans and the plurality of optimization precursor information items as a training corpus.

Those skilled in the art understand that machine learning comprises a branch of artificial intelligence. Machine learning typically employs learning algorithms such as Bayesian networks, decision trees, nearest-neighbor approaches, and so forth, and the process may operate in a supervised or unsupervised manner as desired. Deep learning (also sometimes referred to as hierarchical learning, deep neural learning, or deep structured learning) is a subset of machine learning that employs networks capable of learning from data that is unstructured or unlabeled. Deep learning architectures include deep neural networks, deep belief networks, recurrent neural networks, and convolutional neural networks. Many machine learning algorithms build a so-called “model” based on sample data, known as training data or a training corpus, in order to make predictions or decisions without being explicitly programmed to do so.

By one approach, these teachings provide for generating the machine learning model by, at least in part, selecting a loss function to be minimized during training of the machine learning model.

By one approach, these teachings provide for generating the machine learning model using the plurality of previously-optimized radiation treatment plans and the plurality of optimization precursor information items as a training corpus by, at least in part, evaluating dose distributions in the plurality of previously-optimized radiation treatment plans as a function of the plurality of optimization precursor information items to identify emphasized features. Examples of emphasized features include, but are not limited to, at least one of a feature corresponding to an organ-at-risk protection compromise, a feature corresponding to a compromise between target coverage and organ-at-risk protection, and a feature corresponding to at least one spatially restricted area that has particular weight in achieving or failing a precursor specification. (Note that the foregoing trade-offs may vary considerably from one patient to the next, as such trade-offs may reflect very different patient presentations.)

At optional block 204, this process 200 provides for optimizing a new radiation treatment plan as a function, at least in part, of the foregoing generated machine learning model. At optional block 205, these teachings can provide for administering radiation treatment therapy to a patient 104 as a function of the foregoing new radiation treatment plan.

So configured, these teachings can supplement a machine learning process with information comprising or reflecting features of the training set dose distributions that were emphasized during an original planning process and which features might simply be a side product of an original optimization process. By including such things as clinical goals in the training corpus, the dose distributions in the machine learning can be effectively evaluated to determine those features that were likely intentionally emphasized. So configured, these teachings can leverage the efforts of clinicians for a particular facility to meet local facility protocols.

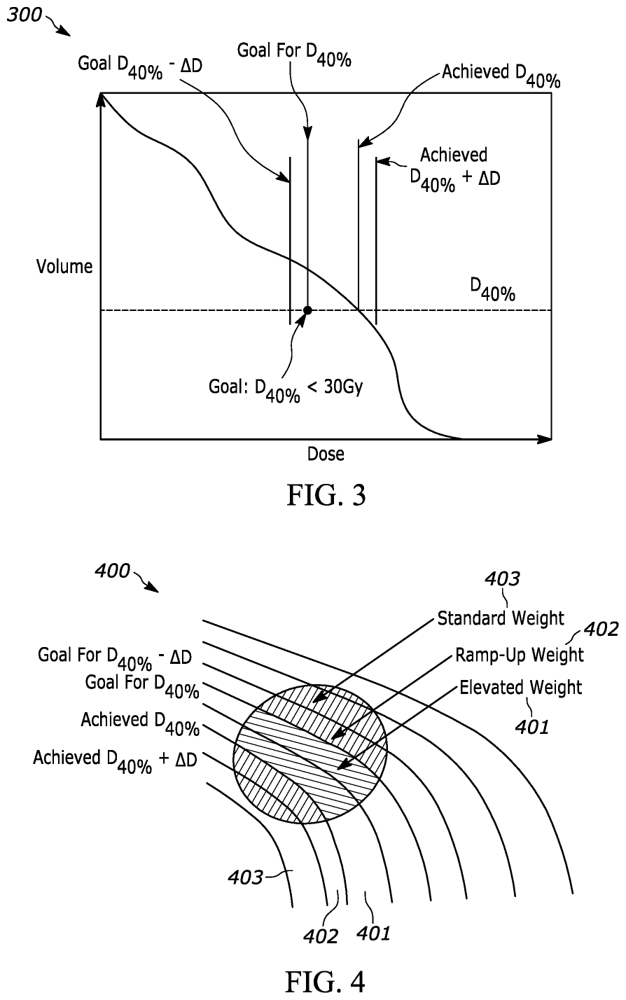

Referring now to FIGS. 3 and 4, an illustrative example that accords with these teachings will be presented. It shall be understood that the specific details of this example are intended to serve an illustrative purpose and are not intended to suggest any particular limitations as regards these teachings.

In this example training of the machine learning model is based on selecting a loss function that is to be minimized during training. In particular, this example presumes the model to comprise a three-dimensional dose prediction model and the loss function can comprise an L2 norm between predicted dose distribution and original clinical dose distribution (for example, a sum of quadratic voxel differences). (Those skilled in the art will know that an L2 norm pertains to vector norms in machine learning where vector norms refer to vector lengths or magnitudes. Generally speaking, an L2 norm is calculated as the square root of the sum of squared vector values. When used in the context of machine learning, use of L2 norms provides regularization that tends to minimize the size of model coefficients and hence model complexity.)

One can add a voxel-specific weight (typically a constant) for any voxel-based loss function. In this example, the weighting is increased for voxels that contribute to features that do not meet clinical goals (including, if desired, features corresponding to clinical goals that are just barely met by a predetermined threshold such as a percentage between, for example, 0% and 5% or 0% and 10%).

More particularly, for the purposes of this example, consider a failing volume-to-dose type of clinical goal (such as “no more than 40% of voxels may receive a dose beyond 30 Gy in the patient's bladder,” a circumstance illustrated in FIG. 3 in graph 300). When this goal fails for one training set plan it likely means that there are too many hot voxels within the organ. It may be noted this circumstance likely reflects a situation with the clinician likely expended additional effort to try and reduce the dose to the achieved level.

By one approach, this failed goal can be associated to individual dose voxels by assuming that any voxel whose dose is higher than the threshold dose level specified in the goal is contributing to the volume of the over-the-threshold dose.

By another approach, this failed goal can be associated to individual dose voxels by assuming that it is the voxels only belonging to the excess volume of the over-the-threshold dose that are contributing to the failing goal and that the most natural selection is the coldest voxels that nevertheless have an over-the-threshold dose level. Dose distributions are often relatively smooth, in that doses in two neighboring voxels cannot usually arbitrarily differ much. This leads naturally to relatively smoothly connected sets of voxels for a given sub-volume. During optimization, however, there are typically contradicting requests for different sub-volumes (for example, in critical organs versus target volumes). The applicant has determined it can be useful to change the plan in a way such that changes are, at least to a reasonable extent, made for those voxels that require a smallest change to a current dose value.

FIG. 4 provides a corresponding illustration in these regards. In this illustration 400, the affected voxels are those where the dose is between the achieved D40% and the goal value (that is, the coldest “too hot” voxels).

Still referring to FIG. 4, it will be noted that some regions 401 are denoted as corresponding to an elevated weight, a next-adjacent region 402 is denoted as corresponding to a ramp-up weight, and a more distant region 403 is denoted as corresponding to a standard weight. The ramp-up weight applies to voxels having a little lower value than the goal value for D40% and those voxels with a little higher value than the achieved value for D40%. Using this approach, one increase the weights of the voxels that fall in the category “coldest among the ones that exceed the goal value.” Additionally, one can also put another weight, although smaller than the one used for the previous category, for those voxels that fall in the ramp-up category. It can be seen that such a ramp-up approach allows a more gradual decrease of the voxel weighting.

These teachings are practical in implementation and will accommodate variations and or supplemental approaches. By one approach, for example, one or more individual items (such as individual optimization precursor information items) can be individually weighted to thereby increase or deemphasize the relative influence of such items as regards training of the machine learning model.

As another example, other strategies can be accommodated to determine the magnitude of the aforementioned increase in weighting. In some cases priorities are associated with particular clinical goals. In such a case, a clinician typically gives more attention to higher-priority goals when it appears that such goals are not being met or are only barely being met. Also, any metric that is close to the goal level is likely to receive more emphasis than those goals that are met by a wide margin. In such a case, the clinicians focus will likely be on other features that require attention to improve performance-to-goal. Furthermore, in a given case where a particular goal is severely missed, a particular organ may have been sacrificed and thus additional dosing was not viewed as representing a severe circumstance.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A method comprising:
by a control circuit:
accessing a plurality of previously-optimized radiation treatment plans;
accessing a plurality of optimization precursor information items, wherein each of the optimization precursor information items corresponds to one of the plurality of previously-optimized radiation treatment plans;
generating a machine learning model using the plurality of previously-optimized radiation treatment plans and the plurality of optimization precursor information items as a training corpus.

2. The method of claim 1 wherein at least one of the optimization precursor information items corresponds to at least two of the plurality of previously-optimized radiation treatment plans.

3. The method of claim 1 wherein at least a majority of the plurality of optimization precursor information items originated with a given radiation treatment facility.

4. The method of claim 3 wherein at least substantially all of the plurality of optimization precursor information items originated with the given radiation treatment facility.

5. The method of claim 1 wherein at least some of the plurality of optimization precursor information items comprise clinical goals.

6. The method of claim 1 wherein at least some of the plurality of optimization precursor information items comprise optimization objectives.

7. The method of claim 1 wherein generating the machine learning model using the plurality of previously-optimized radiation treatment plans and the plurality of optimization precursor information items as a training corpus comprises, at least in part, evaluating dose distributions in the plurality of previously-optimized radiation treatment plans as a function of the plurality of optimization precursor information items to identify emphasized features.

8. The method of claim 7 wherein the emphasized features include at least one of:
a feature corresponding to an organ-at-risk protection compromise;
a feature corresponding to a compromise between target coverage and organ-at-risk protection;
a feature corresponding to at least one spatially restricted area that has particular weight in achieving or failing a precursor specification.

9. The method of claim 1 wherein generating the machine learning model using the plurality of previously-optimized radiation treatment plans and the plurality of optimization precursor information items as a training corpus comprises, at least in part, selecting a loss function to be minimized during training of the machine learning model.

10. The method of claim 1 further comprising:
optimizing a new radiation treatment plan as a function, at least in part, of the machine learning model.

11. An apparatus comprising:
a memory having stored therein:
a plurality of previously-optimized radiation treatment plans; and
a plurality of optimization precursor information items, wherein each of the optimization precursor information items corresponds to one of the plurality of previously-optimized radiation treatment plans;
a control circuit operably coupled to the memory and configured to generate a machine learning model using the plurality of previously-optimized radiation treatment plans and the plurality of optimization precursor information items as a training corpus.

12. The apparatus of claim 11 wherein at least one of the optimization precursor information items corresponds to at least two of the plurality of previously-optimized radiation treatment plans.

13. The apparatus of claim 11 wherein at least a majority of the plurality of optimization precursor information items originated with a given radiation treatment facility.

14. The apparatus of claim 13 wherein at least substantially all of the plurality of optimization precursor information items originated with the given radiation treatment facility.

15. The apparatus of claim 11 wherein at least some of the plurality of optimization precursor information items comprise clinical goals.

16. The apparatus of claim 11 wherein at least some of the plurality of optimization precursor information items comprise optimization objectives.

17. The apparatus of claim 11 wherein the control circuit is configured to generate the machine learning model using the plurality of previously-optimized radiation treatment plans and the plurality of optimization precursor information items as a training corpus by, at least in part, evaluating dose distributions in the plurality of previously-optimized radiation treatment plans as a function of the plurality of optimization precursor information items to identify emphasized features.

18. The apparatus of claim 17 wherein the emphasized features include at least one of:
a feature corresponding to an organ-at-risk protection compromise;
a feature corresponding to a compromise between target coverage and organ-at-risk protection;
a feature corresponding to at least one spatially restricted area that has particular weight in achieving or failing a precursor specification.

19. The apparatus of claim 11 wherein the control circuit is configured to generate the machine learning model using the plurality of previously-optimized radiation treatment plans and the plurality of optimization precursor information items as a training corpus by, at least in part, selecting a loss function to be minimized during training of the machine learning model.

20. The apparatus of claim 11 wherein the control circuit is further configured to:
optimize a new radiation treatment plan as a function, at least in part, of the machine learning model.
